(19) United States
(12) Patent Application Publication
(10) Pub. No.: US 2026/0115128 A1
(43) Pub. Date: Apr. 30, 2026

(54) COSMETIC COMPOSITION FOR SKIN IMPROVEMENT COMPRISING SUBCRITICAL WATER EXTRACT OF ALMOND HULL AS AN ACTIVE INGREDIENT

(71) Applicant: Blessed Project, Paju-si (KR)

(72) Inventors: Yeon Ju BAEK, Cheonan-si (KR); Da Sol AN, Seoul (KR)

(21) Appl. No.: 19/087,465

(22) Filed: Mar. 22, 2025

(30) Foreign Application Priority Data
Oct. 29, 2024 (KR) 10-2024-0149512

(51) Int. Cl. A61K 8/9789; A61Q 17/00; A61Q 19/02

(52) U.S. Cl. CPC A61K 8/9789; A61Q 17/00; A61Q 17/005; A61Q 19/02; A61K 2800/805

(57) ABSTRACT

The present disclosure relates to a cosmetic composition for skin improvement, and more particularly, to a cosmetic composition for skin improvement comprising an almond hull subcritical water extract as an active ingredient. The cosmetic composition according to the present disclosure is a high value-added raw material using environmentally friendly and non-toxic water, and has excellent antioxidant, whitening, and antibacterial activities so that it can effectively improve the skin.

### CROSS-REFERENCE TO RELATED APPLICATION

This application claims the benefit of and priority to Korean Patent Application No. 10-2024-0149512, filed on Oct. 29, 2024, the entire disclosure(s) of which is hereby incorporated herein by reference in its entirety.

### TECHNICAL FIELD

The present disclosure relates to a cosmetic composition for skin improvement, and more particularly, to a cosmetic composition for skin improvement comprising an almond hull subcritical water extract as an active ingredient.

### BACKGROUND

Almond hull is a type of almond byproduct that has been traditionally used as livestock bedding and animal feed. Almond hull is rich in carbohydrates and fiber, and research is ongoing to create added value from it. In particular, attempts are being actively conducted to increase the extraction yield of physiologically active compounds in almond hull in order to minimize almond waste discharge and efficiently utilize almond hull in an environmentally friendly manner, and extraction methods using various solvents have been studied and reported.

Meanwhile, subcritical water refers to water in a high temperature and high pressure state of the critical point of water (374° C., 22.1 MPa) or less. Subcritical water extraction is used to extract useful components of a substance by using the strong hydrolytic power and organic matter dissolving power of subcritical water. Since subcritical water extraction changes depending on temperature and pressure, useful components may be extracted through material transformation by hydrolysis-induced low molecular weight, thermal decomposition, and oxidative decomposition. Subcritical water extraction has merits in that a solvent used is water, and a time required in extraction is short, and also has an advantage in that the extraction yield is improved.

The cosmetics industry is one of the high value-added industries, and recently, interest in and consumption of cosmetics have been increasing regardless of age and gender. In particular, consumption of functional cosmetics that improve skin condition or directly show efficacy on the skin is rapidly increasing. Cosmetics distributed today contain a large amount of harmful chemical ingredients that irritate the skin, so when used on sensitive skin, problems such as skin trouble or skin damage have rather occurred.

In order to solve these problems, the cosmetics industry is actively developing products using natural materials to reduce skin irritation caused by chemicals, etc. Accordingly, the cosmetics industry has recently been making great efforts to develop cosmetics using natural materials that not only have fewer side effects on the skin, but also have excellent biostability on the skin and excellent skin improvement effects. In addition, as consumers' response to cosmetics using natural materials has recently increased, the development value of natural materials as cosmetic raw materials is further increasing.

Accordingly, the inventors of the present disclosure have completed the present disclosure by confirming that the extract has excellent skin improvement effects such as antioxidant effects, antibacterial effects, and whitening effects when almond hull, an eco-friendly material, is extracted using a subcritical water extraction method.

### RELATED ART DOCUMENT

### Patent Document

Korean Patent No. 10-2524053

### SUMMARY

The problem to be solved by the present disclosure is to provide a cosmetic composition for skin improvement comprising an almond hull subcritical water extract as an active ingredient.

In order to solve the above problem, the present disclosure provides a cosmetic composition for skin improvement comprising an almond hull subcritical water extract as an active ingredient.

The composition may exhibit one or more properties selected from the group consisting of antioxidant, whitening, and antibacterial activities.

The subcritical water extraction may be performed at a temperature of 130 to 150° C. and a pressure of 8 to 12 MPa for 10 to 20 minutes.

The composition may comprise the almond hull subcritical water extract at a concentration of 5 to 6.25 mg/ml.

The almond hull subcritical water extract may comprise polyphenols.

### Advantageous Effects

The cosmetic composition according to the present disclosure is a high value-added raw material using environmentally friendly and non-toxic water, and exhibits excellent antioxidant, whitening, and antibacterial activities so that it can effectively improve the skin, and thus can be used as a functional cosmetic in the future. In addition, the amount of almond waste can be minimized by effectively utilizing almond hull, which is an almond by-product generated from almond production and harvesting.

### BRIEF DESCRIPTION OF THE DRAWINGS

FIG. 1 shows a schematic diagram of a subcritical water extractor.

FIG. 2 shows results of measuring the total polyphenol contents of almond hull subcritical water extracts depending on subcritical water extraction temperatures and extraction time conditions.

FIG. 3 shows results of measuring the DPPH radical scavenging activities of almond hull subcritical water extracts depending on subcritical water extraction temperatures and extraction time conditions.

FIG. 4 shows results of measuring the FRAP of almond hull subcritical water extracts depending on subcritical water extraction temperatures and extraction time conditions.

FIG. 5 shows results of measuring the ABTS radical scavenging activities of almond hull subcritical water extracts depending on subcritical water extraction temperatures and extraction time conditions.

FIG. 6 shows cytotoxicities of almond hull subcritical water extracts depending on concentrations (AHE: almond hull subcritical water extract).

FIG. 7 shows whitening effects of almond hull subcritical water extracts depending on concentrations (AHE: almond hull subcritical water extract).

FIG. 8 shows inhibition effects of Staphylococcus aureus depending on the concentrations of almond hull subcritical water extracts.

FIGS. 9, 10, 11 and 12 show plate count agar (PCA) results after treating almond hull subcritical water extracts.

### DETAILED DESCRIPTION

The present disclosure confirmed that when almond hull, an eco-friendly material, is extracted using a subcritical water extraction method, the extract has excellent skin improvement effects such as antioxidant effects, antibacterial effects, and whitening effects.

Therefore, in one aspect, the present disclosure relates to a cosmetic composition for skin improvement comprising an almond hull subcritical water extract as an active ingredient.

In the present disclosure, almond hull is a byproduct generated during the almond production and harvesting process.

In the present disclosure, the term “subcritical water extraction” is a method of decomposing and extracting a substance using ionized water under temperature and pressure conditions of the critical point of water (374° C., 22.1 MPa) or less, and is called pressurized hot water extraction (PHWE) or pressurized low polarity water extraction (PLPW).

In the present disclosure, the term “active ingredient” refers to an ingredient that exhibits the intended activity alone or can exhibit the intended activity together with a carrier or the like that is inactive in itself.

The method for subcritical water extraction comprises steps of: pulverizing almond hull; supplying pulverized almond hull to a subcritical water extractor; and extracting subcritical water.

The step of pulverizing almond hull is a step of finely cutting almond hull with a pulverizer, and almond hull may be pulverized using a pulverizer, a micropulverizer, a blender, a mixer, a fine chopper, etc., but is not limited thereto.

The step of supplying pulverized almond hull to a subcritical water extractor is a previous step for extracting subcritical water. The pulverized almond by-products may be supplied alone, but may be supplied after adding a step of mixing a substance that does not participate in a subcritical water reaction, such as diatomaceous earth, silica, or the like, in order to increase the surface area of the pulverized almond by-products.

The step of extracting subcritical water is a step of extracting polyphenols from the almond by-products by setting temperature and pressure, and subcritical water extraction may be performed by supplying distilled water that is a solvent to a subcritical water extractor and then setting temperature and pressure conditions. At this time, the polyphenol extraction yield, antioxidant activity, antibacterial activity, and whitening activity may vary depending on the temperature, pressure, and time conditions.

In the present disclosure, the subcritical water extraction may be preferably performed at a temperature of 130 to 150° C., and more preferably at 130° C. When the subcritical water extraction temperature is less than the above range, there is a problem in that the polyphenol extraction yield decreases, and when it exceeds the above range, there is a problem in that the thermal stability of polyphenols decreases to change the molecular structure.

In addition, the subcritical water extraction in the present disclosure may be preferably performed at a pressure of 8 to 12 MPa, and more preferably at a pressure of 10 MPa. When the subcritical water extraction pressure is less than the above range, there is a problem in that the vaporization point is reduced, thereby reducing the polyphenol extraction yield, and when it exceeds the above range, there is a problem in that the stability of the polyphenol molecular structure is reduced. In one embodiment of the present disclosure, the subcritical water extraction was performed at a pressure of 10 MPa.

In addition, the subcritical water extraction in the present disclosure may be preferably performed for 10 to 20 minutes, and more preferably for 20 minutes. When the subcritical water extraction time is less than the above range, there is a problem in that the polyphenol cannot be sufficiently extracted, and when it exceeds the above range, there is a problem in that the stability of the polyphenol is reduced.

The cosmetic composition of the present disclosure may exhibit one or more properties selected from the group consisting of antioxidant, whitening, and antibacterial activities.

In one embodiment of the present disclosure, the antioxidant activity was measured using 2,2-diphenyl-1-picrylhydrazyl (DPPH) free radical scavenging assay, ferric reducing antioxidant power (FRAP) assay, and 2,2′-azino-bis(3-ethylbenzothiazoline-6-sulfonic acid) (ABTS) free radical scavenging activity assay methods. As a result, it was confirmed that the antioxidant activity was the most excellent in all of the DPPH assay, FRAP assay, and ABTS assay when almond hull was extracted with subcritical water at 130° C. and 10 MPa for 20 minutes (see Example 2).

Therefore, the subcritical water extraction may preferably be performed at a temperature of 130 to 150° C. and a pressure of 8 to 12 MPa for 10 to 20 minutes, and more preferably at a temperature of 130° C. and a pressure of 10 MPa for 20 minutes.

In one embodiment of the present disclosure, whitening activity was checked by measuring melanin content. When the concentration of the almond hull subcritical water extract was 500 μg/mL or higher, significant cytotoxicity was exhibited, and it was confirmed that the melanin content was lowered when the concentration of the almond hull subcritical water extract was 300 to 500 μg/mL (see Example 3).

The term “antibacterial” in the present disclosure means an action that inhibits or controls the growth and proliferation of microorganisms such as bacteria, fungi, etc., and may mean an action that inhibits or controls the growth and proliferation of acne-causing bacteria and/or skin flora.

The cosmetic composition may have antibacterial activity against one or more strains selected from the group consisting of Staphylococcus aureus, Bacillus subtilis, Escherichia coli, Clostridium perfringens, and Propionibacterium acnes, and preferably have antibacterial activity against Staphylococcus aureus.

“Staphylococcus aureus” is a gram-positive facultative anaerobic bacterium, a pathogen that commonly exists on the skin and nasal surfaces of healthy humans or livestock, and is known to produce heat-resistant exotoxins to cause food poisoning, and secrete phagocyte-killing toxins (leukocidins), hemolysins, coagulase, etc., thereby evading the immune defenses of infected host cells and cause pyogenic infections. In addition, Staphylococcus aureus is commonly found in the skin, nasal cavity, etc. of not less than 50% of healthy people and animals, and may cause mild skin infections such as acne and impetigo.

In the present disclosure, the composition may preferably comprise the almond hull subcritical water extract at a concentration of 5 to 6.25 mg/ml. When the concentration of the almond hull subcritical water extract is less than the above range, the antibacterial activity is weak, and when it exceeds the above range, toxicity may appear on human skin.

In one embodiment of the present disclosure, the antibacterial activity was confirmed through the inhibition effect of Staphylococcus aureus according to the concentration of the almond hull subcritical water extract, and when almond hull was extracted with subcritical water at 130° C. and 10 MPa for 20 minutes, the antibacterial effect began to appear significantly at a concentration of 5 mg/ml, and it was confirmed that it was completely eradicated at 6.25 mg/ml (see Example 4).

In addition, the cosmetic composition may also be prepared in any formulation commonly prepared in the art to which the present disclosure pertains. For example, the composition may be formulated as a solution, a suspension, an emulsion, a paste, a gel, a cream, a lotion, a powder, a soap, a surfactant-containing cleanser, an oil, a powder foundation, an emulsion foundation, a wax foundation, a spray, or the like. Specifically, it may be prepared in the formulation of a toner such as a flexible toner or a nourishing toner, an emulsion such as a facial lotion or a body lotion, a cream such as a nourishing cream, a moisturizing cream, or an eye cream, a foundation such as an essence, a cosmetic ointment, a spray, a gel, a pack, a sunscreen, a makeup base, a liquid type, a solid type, or a spray type, a makeup remover such as a powder, a cleansing cream, a cleansing lotion, or a cleansing oil, a cleanser such as a cleansing foam, a soap, or a body wash, a liquid or gel formulation of mesotherapy that may be injected into the skin layer, and a formulation mixed with a hyaluronic acid gel, but is not limited thereto.

In addition, the cosmetic composition may further comprise additional ingredients commonly used in the cosmetic composition in addition to the almond hull subcritical water extract of the present disclosure, e.g., any conventional cosmetic ingredients that may be selected from purified water, surfactants, excipients, thickeners, dispersants, fillers, preservatives, bactericides, antioxidants, stabilizers, antiseptics, neutralizers, sweeteners, vitamins, free radical scavengers, metal ion sequestrants, fragrances, functional ingredients and mixtures thereof. Those skilled in the art may select any of the additional ingredients and/or their amounts such that the advantageous properties of the composition according to one aspect are not or are not substantially adversely affected by the anticipated addition.

In addition, the cosmetic composition may be appropriately mixed with ingredients commonly used in external skin preparations such as cosmetics, medicines or the like, such as aqueous ingredients, oily ingredients, powder ingredients, alcohols, moisturizers, thickeners, ultraviolet absorbers, whitening agents, antiseptics, antioxidants, surfactants, fragrances, colorants, various skin nutrients, or combinations thereof, as needed.

The cosmetic composition may also be appropriately mixed with metal sequestrants such as sodium edetate, trisodium edetate, sodium citrate, sodium polyphosphate, sodium metaphosphate, gluconic acid, etc.; various herbal medicines such as caffeine, tannin, bellapamil, licorice extract, glablidine, hot water extract of the fruit of Kalin, etc.; drugs such as tocopherol acetate, glycyrrhizic acid, tranexamic acid, derivatives or salts thereof, etc.; and saccharides such as vitamin C, magnesium ascorbic acid phosphate, ascorbic acid glucoside, arbutin, kojic acid, glucose, fructose, trehalose, etc.

In addition, when the cosmetic composition is a solution or emulsion formulation, it may further comprise a solvent, a solvating agent, or an emulsifying agent, such as water, ethanol, isopropanol, ethyl carbonate, ethyl acetate, benzyl alcohol, propylene glycol, glycerol aliphatic ester, polyethylene glycol, sorbitan fatty acid ester, or the like.

In addition, when the cosmetic composition is a suspension formulation, it may further comprise a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar or tragacanth, etc. as a carrier component.

In addition, when the cosmetic composition is a powder or spray formulation, it may further comprise lactose, talc, silica, aluminum hydroxide, calcium silicate or polyamide powder as a carrier component, and particularly in the case of a spray formulation, it may further comprise a propellant such as chlorofluorohydrocarbon, propane/butane or dimethyl ether.

In addition, when the cosmetic composition is a cream or gel formulation, it may further comprise animal oil, vegetable oil, wax, paraffin, starch, cellulose derivatives, polyethylene glycol, silicon, bentonite, silica, talc, zinc oxide, etc. as a carrier component.

In addition, when the cosmetic composition is a surfactant-containing cleansing formulation, it may further comprise, as a carrier component, an aliphatic alcohol sulfate, an aliphatic alcohol ether sulfate, a sulfosuccinic acid monoester, an isethionate, an imidazolinium derivative, a methyl taurate, a sarcosinate, a fatty acid amide ether sulfate, an alkyl amidobetaine, an aliphatic alcohol, a fatty acid glyceride, a fatty acid diethanolamide, a vegetable oil, a linolenic derivative, an ethoxylated glycerol fatty acid ester, etc.

In addition, the cosmetic composition may be used by applying it alone or in duplicate, or by applying it in duplicate with another cosmetic composition other than the cosmetic composition according to one aspect. In addition, all the ingredients included in the cosmetic composition comply with the regulations set by each country. In addition, the cosmetic composition according to one aspect may be used according to a conventional method of use, and the number of times of use may vary depending on the user's skin condition or preference.

Hereinafter, the composition and effect of the present disclosure will be described in more detail through Examples. These Examples are only for illustrating the present disclosure, and the scope of the present disclosure is not limited by these Examples.

### <Experimental Method>

### 1. Pretreatment of Almond Hull

Pre-dried almond hull was ground with a blender, and the ground almond hull particles were sieved so that the size of the ground almond hull particles was 1.0 to 1.4 mm. Thereafter, the ground almond hull was evenly spread on a petri dish, dried at a low temperature of 50° C., and then placed in a glass bottle and stored at room temperature (23 to 28° C.) away from air and direct sunlight. The stored almond hull was dried to a moisture content of 3% or less just before extraction so that extraction thereof was performed.

### 2. Subcritical Water Extraction

Extraction was performed at set temperature conditions (110, 130, 150, 170, and 190° C.) for set times (5, 10, and 20 minutes) using a subcritical water extractor (ASE 350, Dionex, Sunnyvale, CA, USA). Distilled water was used as a solvent. After the extraction was completed, the extract was placed in a glass vial together with nitrogen gas, and after the extract was centrifuged at 3500 rpm for 25 minutes, the supernatant was freeze-dried to adjust the concentration and then used in the experiment.

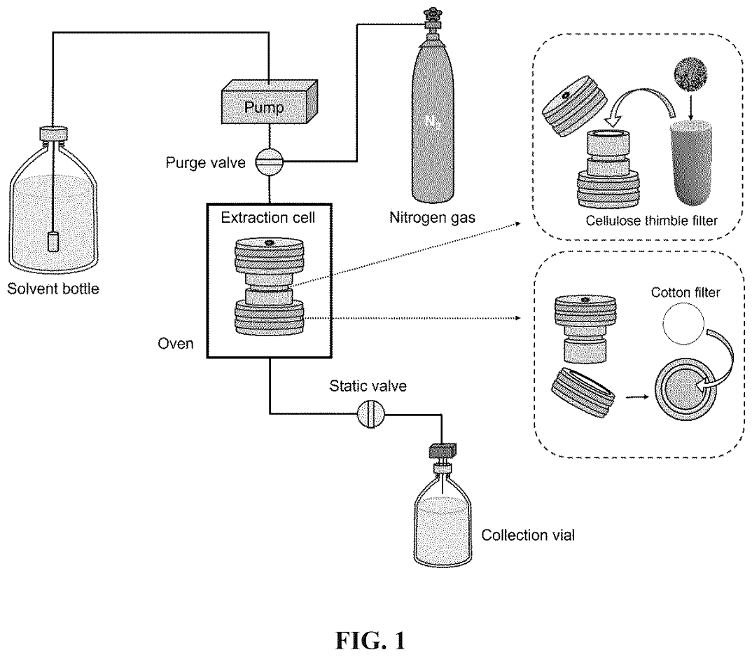

FIG. 1 shows a schematic diagram of a subcritical water extractor, and the specific subcritical water extraction process is as follows. When the temperature of the oven inside the subcritical water extractor reaches a set temperature of the subcritical water extractor, a cell containing an almond hull sample is loaded into the machine, distilled water is filled in the cell together with nitrogen gas, and after applying pressure due to nitrogen gas, the set temperature is maintained, and the static state is maintained for the set extraction time. The pressure was fixed at approximately 10 MPa (≈100 ATM) during performance of the extraction.

### 3. Measurement of Total Polyphenol Content

The total polyphenol content was measured using the folin-ciocalteu method. Specifically, 2 mL of a 2% sodium carbonate (Na₂CO₃) aqueous solution and 0.1 mL (1 mg/mL) of an almond hull extract extracted using a subcritical water extractor were mixed. After vortexing for 3 minutes, 0.1 mL of a 50% folin-ciocalteu reagent was added to the mixed solution, vortexed again, wrapped in aluminum foil, and subjected to dark reaction at room temperature for 30 minutes. After 30 minutes, absorbance was measured at 708 nm using a UV spectrophotometer, and a standard curve was created using gallic acid as a standard reagent. Total polyphenol content was calculated by substituting it into the standard curve. At this time, the unit is gallic acid equivalent (GAE) per 1 g of the weight of dried almond hull.

### 4. Measurement of Antioxidant Activity

Antioxidant activity was measured using 2,2-diphenyl-1-picrylhydrazyl (DPPH) free radical scavenging assay, ferric reducing antioxidant power (FRAP) assay, and 2,2′-azino-bis(3-ethylbenzothiazoline-6-sulfonic acid) (ABTS) free radical scavenging activity assay methods.

### (1) DPPH Free Radical Scavenging Assay

14.2 mg of DPPH (Sigma Aldrich) was put and dissolved in 300 ml of ethanol, and then mixed with an equal amount of distilled water to create a 50% ethanol phase (final concentration=6×10⁻⁵ M). 3 ml of the prepared DPPH solution and 0.1 ml (0.5 mg/ml) of the prepared almond hull extract were mixed, vortexed for several seconds, and then subjected to a dark reaction for 20 minutes. After 20 minutes, 1 ml of the above reaction solution was placed in a Cuvet (PS material) to measure the absorbance at 517 nm using a UV spectrophotometer. A standard curve was created using ascorbic acid as a standard sample. After calculating by substituting it into the standard curve, the DPPH radical scavenging activity was expressed as ascorbic acid equivalent (AAE) mg/g dried almond hull.

### (2) FRAP Assay

10 mM of TPTZ was mixed with a 300 mM sodium acetate buffer (pH 3.6) and 40 mM of hydrochloric acid, and then the mixtures were mixed with a 20 mM FeCl₃·6H₂O solution at a ratio of 10:1:1 to prepare a FRAP reagent. Specifically, the process of preparing a FRAP solution follows steps \textcircled{1} to \textcircled{3} below.

\textcircled{1} 100 ml of 300 mM sodium acetate buffer (pH 3.6):
Lower the pH with sodium acetate hypohydrate 4.0824 g 70 ml+acetic acid, then add distilled water until the volume becomes 100 ml
   \textcircled{2} 20 ml of 20 mM ferric chloride solution:
108.12 mg of FeCl₃·6H₂O+20 ml of distilled water
   \textcircled{3} 10 mM TPTZ solution+40 mM hydrochloric acid:
62. 466 mg of sample+20 ml of 40 mM hydrochloric acid

265 μl of the FRAP solution prepared in a 96-well plate and 35 μl of the prepared almond hull extract (0.5 mg/ml) were added to each well, and a dark reaction was performed at 37° C. for 30 minutes. After 30 minutes, the well plate was placed in a spectrophotometer, shaken for 5 seconds, and the absorbance was measured at 595 nm. A standard curve was created using ascorbic acid as a standard sample. After calculating by substituting it into the standard curve, FRAP assay was performed. The scavenging activity was expressed as AAE mg/g dried almond hull.

### (3) ABTS Free Radical Scavenging Activity Assay

25 ml of a 7 mM ABTS solution and 25 ml of a 2.45 mM potassium persulfate solution were mixed and subjected to a dark reaction at room temperature for 16 hours. The process for preparing an ABTS mixed solution follows steps \textcircled{1} to \textcircled{2} below.

### \textcircled{1} 25 ml of 7 mM ABTS:

0.096 g of 2,2′-azino-bis(3-ethylbenzothiazoline-6-sulfonic acid) diammonium salt (≥98% (HPLC))+25 ml of distilled water
   \textcircled{2} 20 ml of 20 mM ferric chloride solution:
0.0166 g of potassium persulfate+20 ml of distilled water

After the ABTS mixture solution prepared above was filtered through filter paper (Whatman 42, ashless), 1 ml of the filtered ABTS mixture solution was placed in Cuvet, and distilled water was added to dilute it until the absorbance at 734 nm was 1.5 (±0.1). 180 μl of the ABTS solution whose absorbance had been adjusted and 20 μl (0.5 mg/ml) of the prepared almond hull subcritical water extract were placed in each well of a 96-well plate, and the reaction was performed at room temperature for 6 minutes. After 6 minutes, the well plate was placed in a spectrophotometer, shaken for 5 seconds, and then the absorbance was measured at 734 nm. A standard curve was created using ascorbic acid as a standard sample. After calculation by substituting it into the standard curve, assay was performed, and the scavenging activity was expressed as AAE mg/g dried almond hull.

### 5. Measurement of Whitening Activity

### \textcircled{1} Cell Culture

The cells were cultured under conditions of 37° C. and 5% CO₂ by adding 10% fetal bovine serum (FBS, 16000-044) and 1% penicillin-streptomycin (Pen Strep, 15140-122) to Dulbecco's Modified Eagle's Medium (DMEM).

### \textcircled{2} Cell Viability Analysis (CCK-8 Assay)

B16F10 cells were inoculated in 96-well plates at a density of 1×10³ cells/well and cultured for 24 hours. Afterwards, the cells were treated with DMEM containing an almond hull subcritical water extract at an appropriate concentration (0 to 2000 μ/mL) and then cultured for 48 hours. The effect of the almond hull subcritical water extract on B16F10 cell viability was evaluated using the cell counting kit-8 assay according to the manufacturer's instructions.

### \textcircled{3} Measurement of Melanin Content

B16F10 cells were inoculated in 6-well plates at a density of 5×10⁴ cells/well. After 4 hours, the cells were stimulated with IBMX (50 μM) and then treated with an almond hull subcritical water extract for 72 hours. After removing the medium and washing them with PBS, the cells were collected using a cell scraper and then centrifuged at 13,000 rpm for 1 min. The cell pellet was dissolved with 1 N NaOH in 10% DMSO and reacted at 80° C. for 1 hour. The absorbance was measured at 405 nm, and the protein content was estimated by BCA analysis. The melanin content was normalized with respect to total protein and then expressed as a percentage of the untreated control group.

### 6. Measurement of Antibacterial Activity

90 μl of Muller Hilton Broth (MHB) and 90 μl of a serially diluted almond hull subcritical water extract (0.125, 0.625, 1.25, 2.5, 3, 3.5, 4, 4.5, 5, 6.25, 12.5, 25, 50, or 100 mg/ml) were added to each well of a 96-well plate. At this time, distilled water as a negative control group and ampicillin as a positive control group were treated in the same volume together with 90 μl of MHB. After that, 10 μl of bacterial suspension (10⁶ CFU/ml) was added to each well, and Staphylococcus aureus (ATCC25923) was used as the bacteria. After this, it was cultured for 24 hours in an incubator at 37° C., and after 24 hours, 10 μl of resazurin (6.75 mg/ml) was added to the wells containing the extract and the control groups. After reacting for 4 hours in an incubator at 37° C., the well plate was taken out, the absorbance was measured at 500 nm, and then the color was checked to confirm the minimum inhibition concentration (MIC).

At this time, if the color of the resazurin pigment was pink, it was judged that there were bacteria, and if it remained dark blue, it was judged that there were no bacteria. The lowest concentration that maintained the blue color and the concentrations above it were spread on plate count agar (PCA) to check the bacterial count, and the concentration at which all bacteria were eradicated was set as the minimum bactericidal concentration (MBC).

The color of resazurin alone was obtained by subtracting the color absorbance of the sample from the measured absorbance, thereby obtaining the inhibition rate (%) by substituting the absorbance of the sample (ΔAₛₐₘₚₗₑ), the absorbance of the negative control group (ΔA_{neg}), and the absorbance of the positive control group (ΔAₚₒₛ) into the formula below.

$$Inhibition Rate ( \% ) = \frac{Δ {A}_{neg} - Δ {A}_{sample}}{Δ {A}_{neg} - Δ {A}_{pos}} \qquad [ Mathematical Formula 1 ]$$

### [Example 1] Measurement of Total Polyphenol Content of Almond Hull Subcritical Water Extract Depending on Subcritical Water Extraction Temperature and Extraction Time Conditions

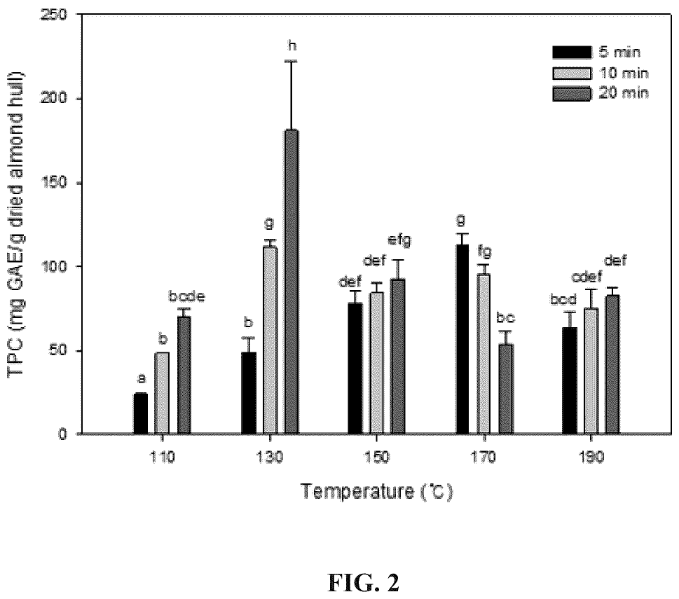

FIG. 2 shows results of measuring total polyphenol contents of almond hull subcritical water extracts depending on subcritical water extraction temperatures and extraction time conditions (unit: mg GAE/g dried almond hull).

Examining FIG. 2, it can be confirmed that the polyphenol content is low when the extraction time is 5 minutes, but the polyphenol content is generally high when the extraction time is 10 to 20 minutes. In addition, even when the extraction time is 10 to 20 minutes, the polyphenol content is about 70 mg GAE/g dried almond hull or less at a low temperature of 110° C., whereas the total polyphenol content in almond hull is high at a maximum of about 181 mg GAE/g dried almond hull at 130 to 150° C.

### [Example 2] Measurement of Antioxidant Activity of Almond Hull Subcritical Water Extract Depending on Subcritical Water Extraction Temperature and Extraction Time Conditions

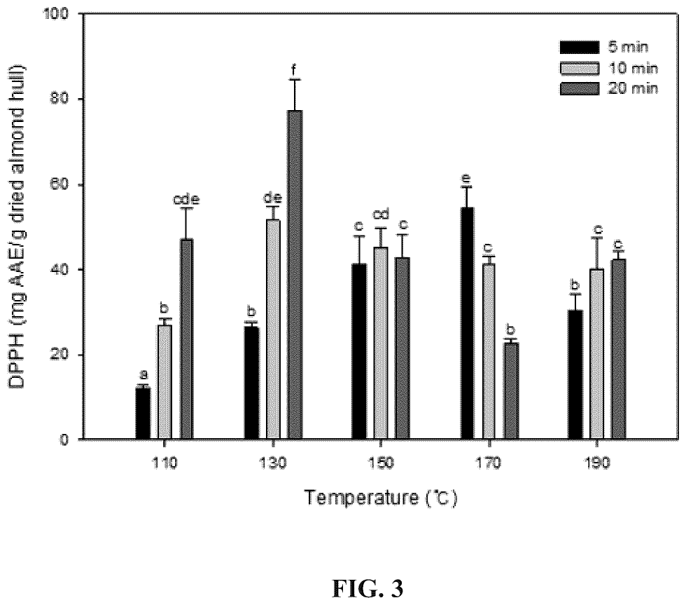

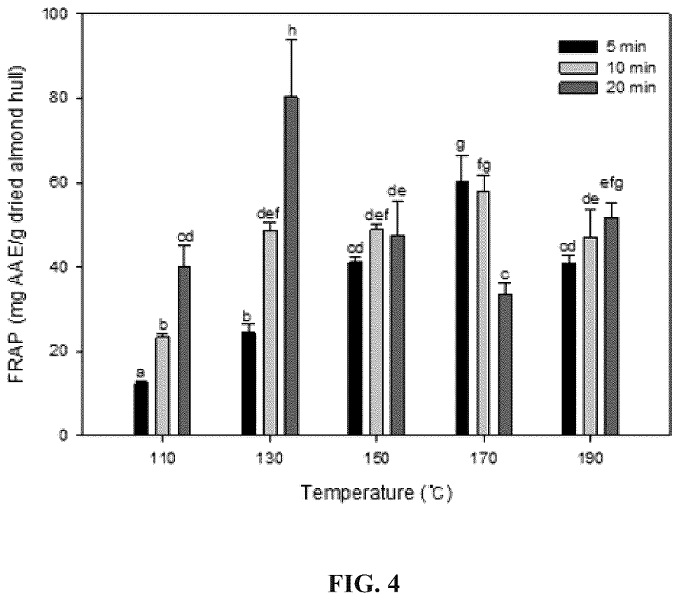

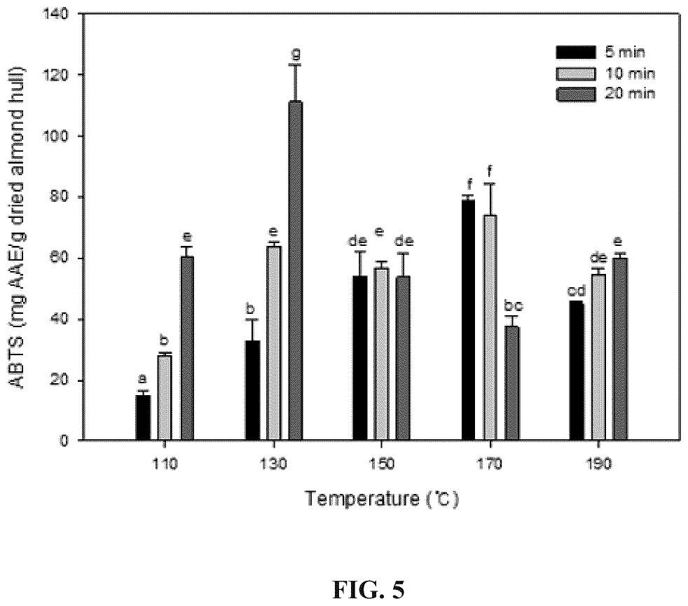

FIGS. 3 to 5 show results of measuring antioxidant activities depending on subcritical water extraction temperature and extraction time conditions of almond hulls, FIG. 3 shows results of measuring DPPH radical scavenging activities, FIG. 4 shows results of measuring FRAP, and FIG. 5 shows results of measuring ABTS radical scavenging activities.

Examining FIG. 3, it can be confirmed that in the DPPH assay, when subcritical water was extracted at 110° C. for 5 minutes, the antioxidant activity was low at about 12 mg AAE/g dried almond hull, but when subcritical water was extracted at 130° C. for 20 minutes, the antioxidant activity was high at about 77 mg AAE/g dried almond hull. In addition, examining FIG. 4, it can be confirmed that in the FRAP assay, when subcritical water was extracted at 110° C. for 5 minutes, the antioxidant activity was low at about 12 mg AAE/g dried almond hull, but when subcritical water was extracted at 130° C. for 20 minutes, the antioxidant activity was high at about 80 mg AAE/g dried almond hull. Also, examining FIG. 5, it can be confirmed that in the ABTS assay, when subcritical water was extracted at 110° C. for 5 minutes, the antioxidant activity is low at about 15 mg AAE/g dried almond hull, but when subcritical water was extracted at 130° C. for 20 minutes, the antioxidant activity was high at about 111 mg AAE/g dried almond hull. Through this, it can be confirmed that the antioxidant activity is the most excellent when the subcritical water extraction condition is 130° C. for 20 minutes.

### [Example 3] Measurement of Whitening Effect of Almond Hull Subcritical Water Extract

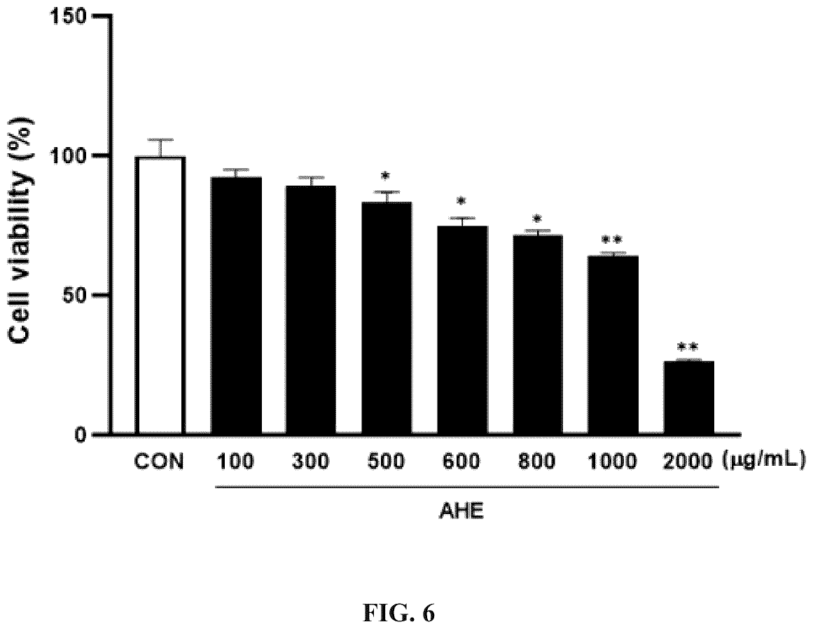

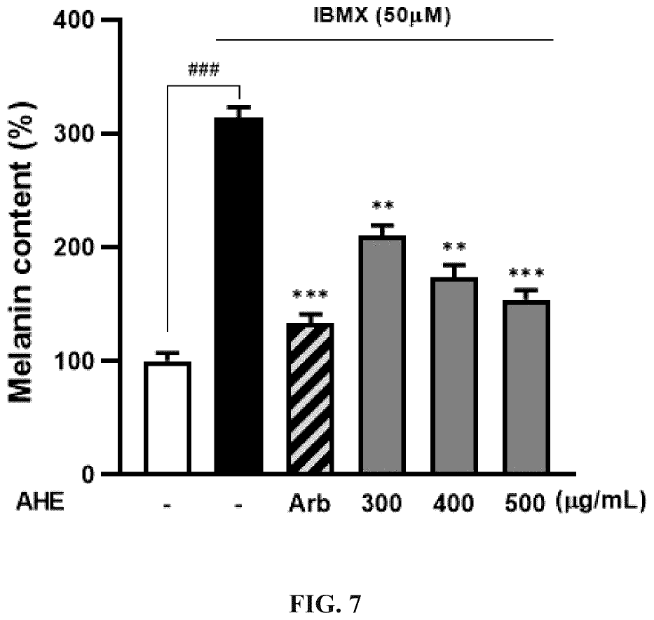

FIG. 6 shows cytotoxicities of almond hull subcritical water extracts depending on concentrations, and FIG. 7 shows whitening effects of almond hull subcritical water extracts depending on concentrations.

Examining FIG. 6, it can be confirmed that significant cytotoxicities occur at concentrations of 500 μg/mL or higher. Accordingly, after the maximum concentration of the almond hull critical water extracts was set to 500 μg/mL, the whitening effects were checked.

As a result, examining FIG. 7, it was confirmed that the melanin contents of the almond hull subcritical water extracts decreased at a concentration of 300 to 500 μg/mL, which indicates that the almond hull subcritical water extracts have whitening effects.

### [Example 4] Measurement of Antibacterial Activity of Almond Hull Subcritical Water Extract

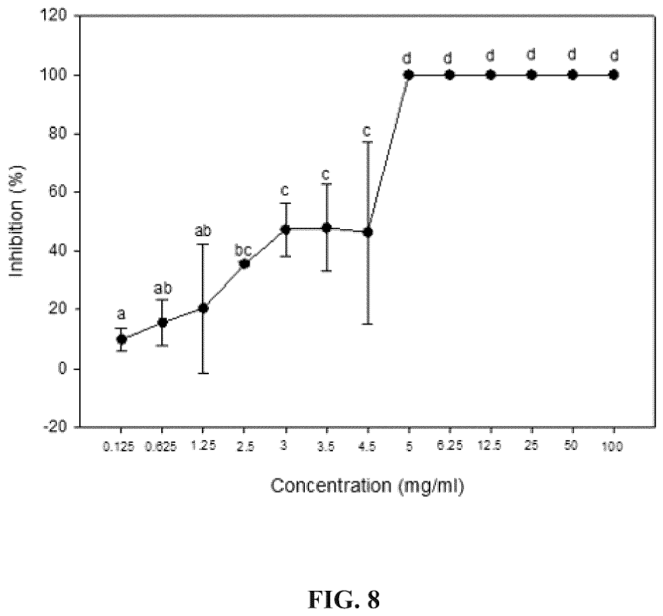

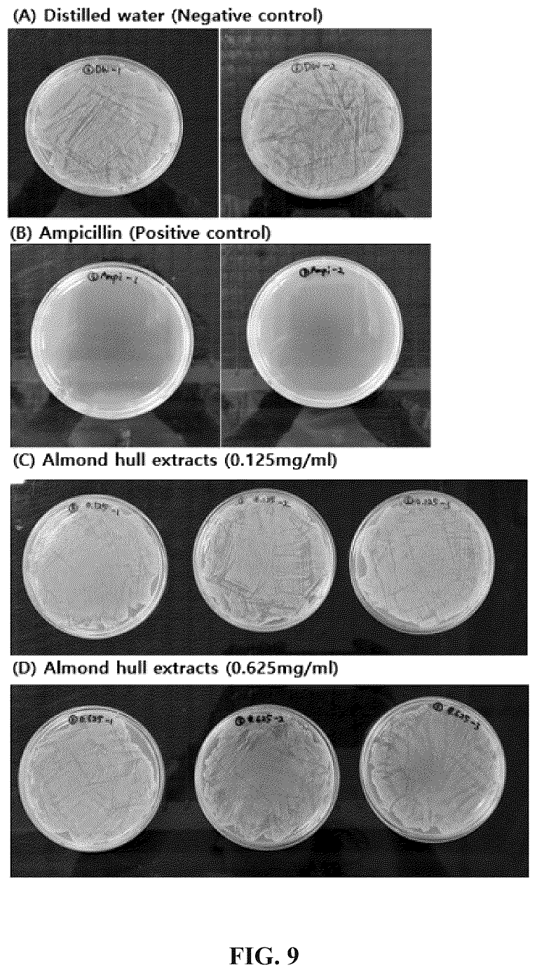

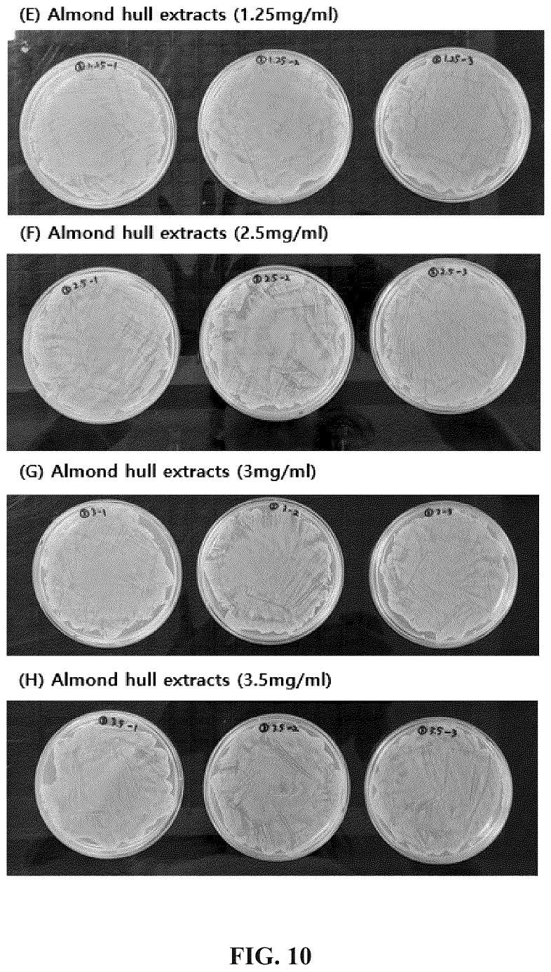

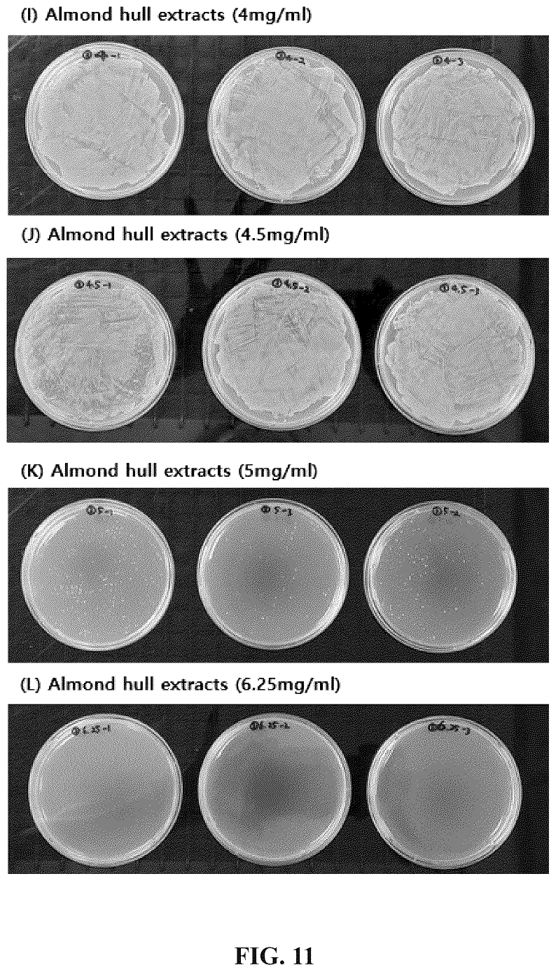

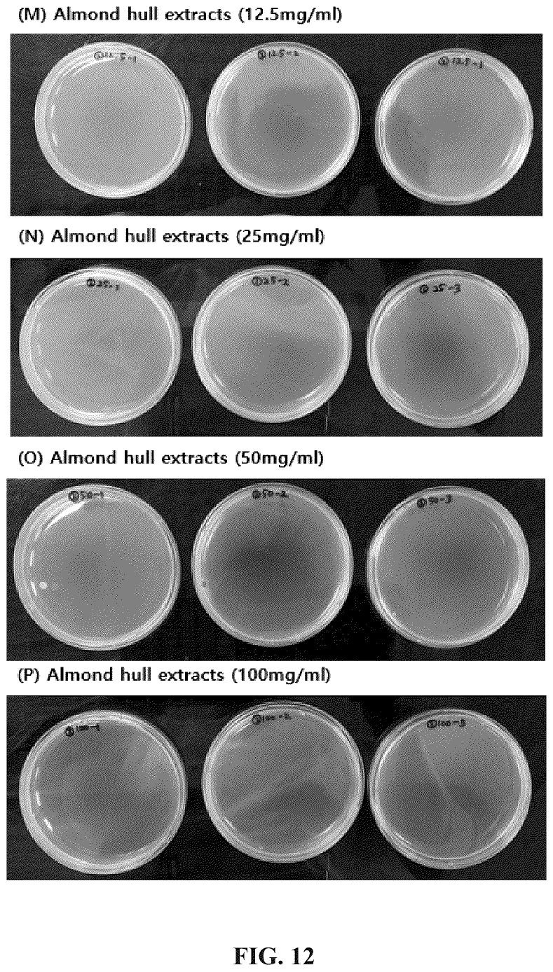

FIG. 8 shows inhibition effects of Staphylococcus aureus depending on the concentrations of almond hull subcritical water extracts, and FIGS. 9 to 12 show plate count agar (PCA) results after treating almond hull subcritical water extracts.

Examining FIG. 8, when subcritical water was extracted at 130° C. for 20 minutes, the lowest concentration (MIC) among the concentrations at which the color of resazurin did not change was 5 mg/mL. Also, examining FIGS. 9 to 12, as results of performing spreading at each concentration in order to check the bacterial count, it was confirmed that the bacteria were completely eradicated at 6.25 mg/ml, and through this, it was confirmed that the minimal bactericidal concentration (MBC) was 6.25 mg/ml. Therefore, it can be seen that the subcritical water extract of almond hull at 130° C. for 20 minutes began to exhibit a significant antibacterial effect at a concentration of 5 mg/ml when the bacterial count was around 6 logs, and completely eradicated the bacteria at 6.25 mg/ml.

Although the present disclosure has been described with reference to Examples, these are merely exemplary, and those skilled in the art to which the present disclosure pertains will understand that various modifications and equivalent other embodiments are possible therefrom. Accordingly, the disclosed Examples should be considered from an illustrative rather than a restrictive perspective, and the true technical protection scope of the present disclosure should be determined by the technical idea of the claims.

What is claimed is:

1. A method for improving skin condition of a subject in need thereof, comprising administering a composition comprising an almond hull subcritical water extract as an active ingredient.

2. The method according to claim 1, wherein the composition has one or more activities selected from the group consisting of antioxidant, whitening, and antibacterial.

3. The method according to claim 1, wherein the subcritical water extraction is performed at a temperature of 130 to 150° C. and a pressure of 8 to 12 MPa for 10 to 20 minutes.

4. The method according to claim 1, wherein the composition comprises the almond hull subcritical water extract at a concentration of 5 to 6.25 mg/ml.

5. The method according to claim 1, wherein the almond hull subcritical water extract comprises polyphenols.

6. The method according to claim 1, wherein the composition is formulated in a form selected from the group consisting of a solution, a suspension, an emulsion, a paste, a gel, a cream, a lotion, a powder, a soap, a surfactant-containing cleanser, an oil, a powder foundation, an emulsion foundation, a wax foundation, and a spray.

7. The method according to claim 1, wherein the composition further comprises one or more ingredients selected from the group consisting of purified water, surfactants, excipients, thickeners, dispersants, fillers, preservatives, bactericides, antioxidants, stabilizers, antiseptics, neutralizers, sweeteners, vitamins, free radical scavengers, metal ion sequestrants, fragrances, and functional ingredients.
